(19) United States
(12) Patent Application Publication
(10) Pub. No.: US 2005/0085798 A1
(43) Pub. Date: Apr. 21, 2005

(54) Adjustable surgical cutting instrument and cam system for use in same

(76) Inventors: Ronald L. Hofmann, Brazil, IN (US); Robert W. Davignon, Terre Haute, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 10/941,244

(22) Filed: Sep. 15, 2004

Related U.S. Application Data
(60) Provisional application No. 60/503,056, filed on Sep. 15, 2003.
(60) Provisional application No. 60/580,068, filed on Jun. 16, 2004.

(51) Int. Cl. A61B 17/00

(52) U.S. Cl. 606/1

(57) ABSTRACT

An adjustable cam and follower system for a surgical device that includes a tri-dimensional rotatable cam member having a rotational input and a cam surface, the contour of which is defined by at least one radius of varying length. The system further includes a follower selectively moveable linearly, the follower configurable to ride substantially upon the cam surface as the cam rotates. A handpiece having an ergonomic member attachable thereto is also disclosed.

### CROSS REFERENCE TO RELATED APPLICATION

This application claims priority to U.S. provisional application 60/580,068 filed on Jun. 16, 2004 and U.S. provisional application 60/503,056 filed on Sep. 15, 2003, both of which are incorporated herein by reference in their entirety.

### BACKGROUND OF THE INVENTION

1. Field of the Invention

The present invention generally relates to the field of surgical cutting instruments and, more particularly, to surgical instruments having a selectively adjustable reciprocating cutter.

2. Description of the Related Art

Cam systems are often used in machines to convert rotary motion into linear motion. One type of cam system uses a rotating cam to linearly drive a follower. In a particular implementation, the follower is held in constant contact with a surface of the cam by a biasing force, typically applied by a spring or an air cylinder. The shape of the rotating cam is generally configured to control the linear motion of the follower. The cam is typically oblong with respect to its rotational axis and has “high” and “low” points on a surface thereof. When the cam rotates to a “low” point or position, the biasing force pushes the follower toward the cam and the center of rotation. In contrast, when the cam rotates to a “high” point or position, the follower is extended away from the center of rotation, compressing the spring. Thus, a continuously rotating cam may be used to produce a linear reciprocating motion in the follower.

In surgical cutting instruments for excising tissue, an elongated handpiece is generally included. A typical handpiece is cylindrical in design and is intended to be held by a surgeon in the same manner as a pencil. Within the surgical cutting instrument cam systems are typically used to reciprocatingly drive a surgical cutting member. In a typical instrument design, the cam is rotatively driven by a motor that resides in the handpiece of the instrument. Unfortunately, the cam systems in conventional surgical cutting instruments are limited in that the operating plane of the surgical cutting member is fixed in relation to the rotational axis of the cam. In other words, the angle of the surgical cutting member cannot be adjusted relative to the handpiece. Among other limitations, the inability to adjust the angle between the surgical cutting member and the handpiece may impair a surgeon's visual access to the surgical site. Furthermore, the inability to modify the cutting member angle relative to the handpiece precludes a surgeon from adjusting the surgical instrument to ergonomically fit his/her hand. Moreover, although the method of holding the handpiece in the same manner as a pencil is intuitive, it does not provide the best control when manipulating the handpiece during long and/or awkward surgical procedures.

In an attempt to improve control, various surgical instruments have been developed having a tapered handpiece and/or other features, such as ridges, knurls or bumps, that facilitate a surgeon's grip on the handpiece. While these features may improve a surgeon's ability to hold the surgical device, they fail to alleviate hand fatigue that may complicate and/or lengthen the surgical procedure. Accordingly, an improved surgical cutting instrument is desired that provides a surgeon with the ability to adjust the angle of a cam driven reciprocating cutting member relative to a handpiece of the instrument and reduce hand fatigue associated with the conventional surgical cutting instruments.

### SUMMARY

An adjustable cam and follower system for a surgical device is provided that includes a tri-dimensional rotatable cam member having a rotational input and a cam surface, the contour of which is defined by at least one radius of varying length. The system further includes a follower selectively moveable linearly, the follower configurable to ride substantially upon the cam surface as the cam rotates. Accordingly, the cam and follower system produces smooth reciprocating movement in the follower. In an embodiment, the cam and follower system is contained within a handpiece having an ergonomic member attachable thereto. The cam and follower system improves ergonomics for a surgeon by enabling the surgeon to adjust the angle of the surgical device thereby modifying the cutting angle of the surgical device. Furthermore, the ergonomic member enables a reduction in hand fatigue by enabling better control when manipulating the surgical device during surgical procedures.

### BRIEF DESCRIPTION OF THE DRAWINGS

Embodiments of the invention will now be described, by way of example, with reference to the accompanying drawings, wherein:

FIG. 1 is a schematic illustration of a cam surface according to an embodiment of the invention.

FIG. 2 is a cross-sectional view of a rotatable cam member according to an embodiment of the invention.

FIG. 3 is a cross-sectional view of a rotatable cam member according to another embodiment of the invention.

FIG. 4 is a cross-sectional view of a rotatable cam member according to another embodiment of the invention.

FIGS. 5A and 5B are cross-sectional views of a rotatable cam member according to another embodiment of the present invention.

FIGS. 6A and 6B are partial cross-sectional views of a cam and follower system according to an embodiment of the present invention.

FIGS. 7A and 7B are partial cross-sectional views of a cam and follower system according to another embodiment of the present invention.

FIGS. 8A-8C are partial cross-sectional views of a fixture demonstrating the adjustment of the follower angle in a cam and follower system according to an embodiment of the present invention.

FIG. 9 is a partial cross-sectional view of a surgical cutting instrument employing a cam and follower system according to an embodiment of the present invention.

FIG. 10 is an illustration of a prior art intracranial surgical handpiece used in a medical procedure.

FIG. 11 is an illustration of a surgical handpiece used in a medical procedure employing the cam and follower system according to an embodiment of the present invention.

FIG. 12 is a side view of a surgical device including an ergonomic attachment according to an embodiment of the present invention.

FIG. 13 is a side view of the ergonomic attachment of FIG. 12.

FIG. 14 is a front view of the ergonomic attachment of FIG. 12.

FIG. 15 is a perspective view of the surgical device and ergonomic attachment of FIG. 12, shown in a surgeon's hand.

FIG. 16 is a side view of a surgical device including an ergonomic attachment according to another embodiment of the present invention.

FIG. 17 is a front view of the ergonomic attachment of FIG. 16.

FIG. 18 is perspective view of the ergonomic attachment of FIG. 16.

FIG. 19 is a perspective view of the surgical device and ergonomic attachment of FIG. 16, shown in a surgeon's hand.

### DETAILED DESCRIPTION

Referring now to the drawings, the illustrative embodiments of the present invention are shown in detail. Although the drawings represent some preferred embodiments of the present invention, the drawings are not necessarily to scale and certain features may be exaggerated to better illustrate and explain the present invention. Further, the embodiments set forth herein are not intended to be exhaustive or otherwise limit or restrict the invention to the precise forms and configurations shown in the drawings and disclosed in the following detailed description.

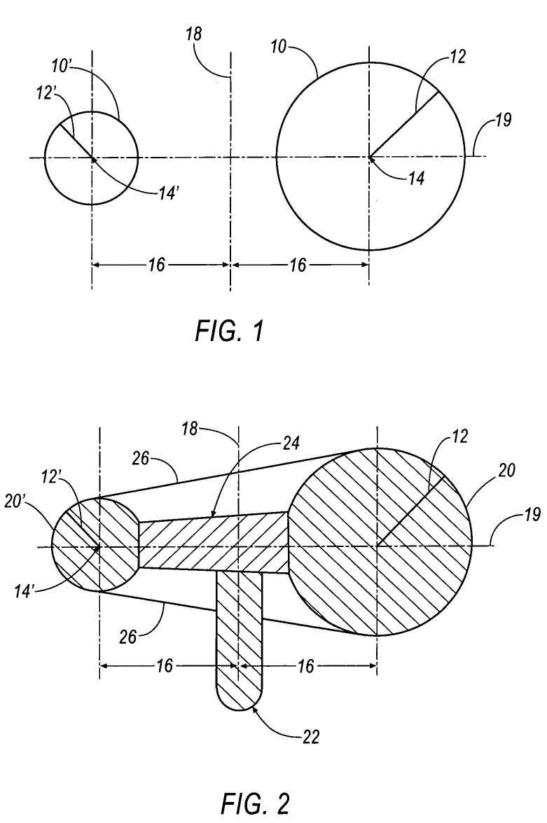

Referring to FIG. 1, a schematic representation of a cam surface 10, 10′ is shown according to an embodiment of the present invention. The surface 10, 10′ of the cam is defined by a radius 12, 12′ rotated about a central surface point 14, 14′, which is shown as being two different points in FIG. 1 but is actually a ring having a radius 16. In this regard, central surface point 14, 14′ is displaced at a fixed distance 16 from the cam's axis of rotation 18 along a plane 19 that is perpendicular to axis 18. Accordingly, radius 12, 12′ varies as central surface point 14, 14′ is rotated about axis 18.

Referring to FIG. 2, a cross-sectional view of a rotatable cam member according to an embodiment of the present invention is shown. The rotatable cam member includes a cam surface 20, 20′, a stem 22 and a hub 24 connecting cam surface 20, 20′ to stem 22. Cam surface 20, 20′ also includes upper and lower transition surfaces 26, which taper according to the contour of cam surface 20, 20′ as radius 12, 12′ is varied.

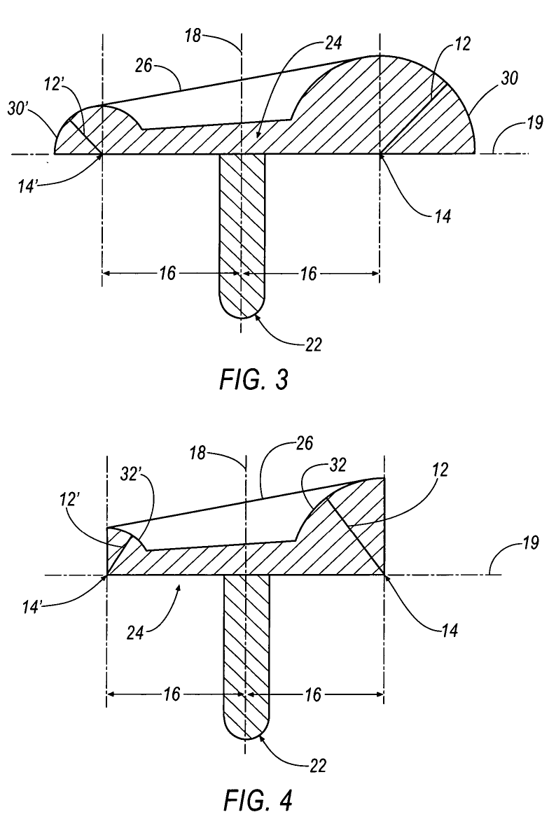

Referring to FIG. 3 a cross-sectional view of a rotatable cam member according to another embodiment of the invention is shown. The illustrated embodiment is substantially similar to the embodiment shown in FIG. 2 with at least one exception, namely, cam surface 30, 30′ does not extend below plane 19.

Referring to FIG. 4 a cross-sectional view of a rotatable cam member according to another embodiment of the invention is shown. The illustrated embodiment is substantially similar to the embodiment shown in FIG. 2 with at least one exception, namely, cam surface 32, 32′ does not extend below plane 19 and does not extend radially outwardly from axis 18 a distance greater than radius 16.

In FIGS. 2-4, transition surface 26 illustrates how radius 12 is smoothly reduced to radius 12′ as central surface point 14, 14′ is rotated about axis 18. However, the invention is not limited to the embodiments shown in FIGS. 2-4. Alternatively, radius 12, 12′ may vary in length and/or contour as central surface point 14, 14′ is rotated about axis 18 to provide a cam member having the desired cam surface profile.

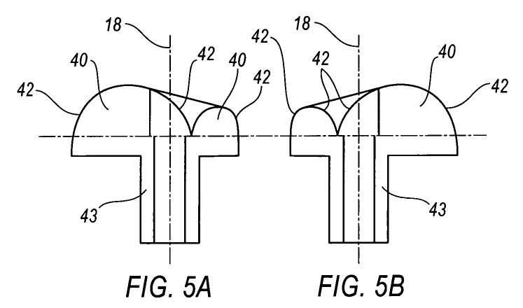

Referring to FIGS. 5A-5B, a rotatable cam member is shown according to another embodiment of the invention. In the illustrated embodiment, the rotatable cam member includes a body 40 having a cam surface 42 and an axis of rotation 18. Cam body 40 may include a stem 43 configured for attachment to the rotational output of a drive member, such as a motor or transmission. Surface 42 is similar to surface 30, 30′ described above in reference to FIG. 3 and is configured to interface with a follower, as will be described below. FIG. 5A shows the rotatable cam member in a first angular position and FIG. 5B shows the rotatable cam member in a second angular position about 180 degrees opposite the first position.

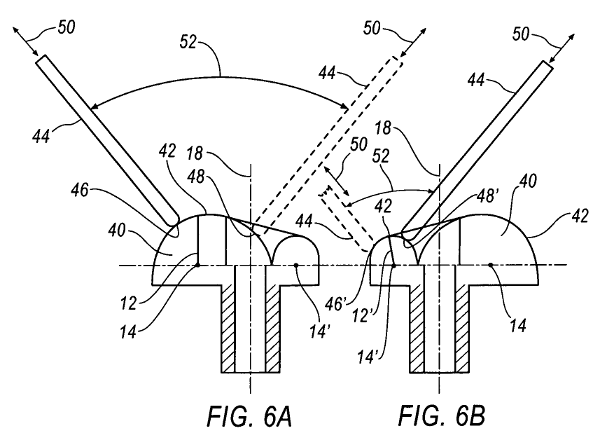

Referring to FIGS. 6A and 6B, a cam and follower system according to an embodiment of the invention is shown. The cam and follower system includes a selectively adjustable follower 44 shown in contact with cam surface 42. In each of FIGS. 6A and 6B, follower 44 is shown in two positions. In either position, follower 44 is free to translate along the axis of the follower, but is otherwise prohibited from moving relative to the rotatable cam member.

FIG. 6A shows the cam body 40 in a first angular position. In this position, follower 44 is shown contacting cam surface 42 at exterior contact point 46 and at an interior contact point 48. Contact points 46 and 48 are in a region of cam body 40 where the outer portion of cam surface 42 is defined by the longest radius 12 and thus, follower 44 is generally at a distance equal to radius 12 from central surface point 14.

FIG. 6B shows the cam body 40 in a second angular position rotated about 180 degrees about the axis of rotation 18. Follower 44 is shown in FIG. 6B as contacting the cam surface 42′ at exterior surface point 46′ and at interior surface point 48′. At this angle of rotation, cam surface 42′ is defined by the shortest radius 12′. When cam body is rotated to the second angular position shown in FIG. 6B, the tip of follower 44 is closer to central surface point 14′ than it was to central surface point 14 when cam body 40 was in the first angular position shown in FIG. 6A. Thus, as cam body 40 is rotated, follower 44 translates reciprocatingly along the axis of the follower to produce a reciprocating output 50. The distance of reciprocation is roughly the difference in length between radius 12 and radius 12′. The cam surface between radii 12 and 12′ is defined by a continuously smooth transition as cam body 40 is rotated about axis of rotation 18, which produces smooth reciprocating movement in follower 44. 1

As noted above, follower 44 may be moved about central surface point 14, 14′ along an arc 52. Since cam surface 42, 42′ is defined by the rotation of radius 12, 12′ about central surface point 14, 14′, the position of follower 44 may be moved about an arc 52 without substantially changing the characteristics of reciprocating output 50. Thus, the position of follower 44 relative to cam body 40 is adjustable without significantly changing the stroke length of the reciprocating output 50.

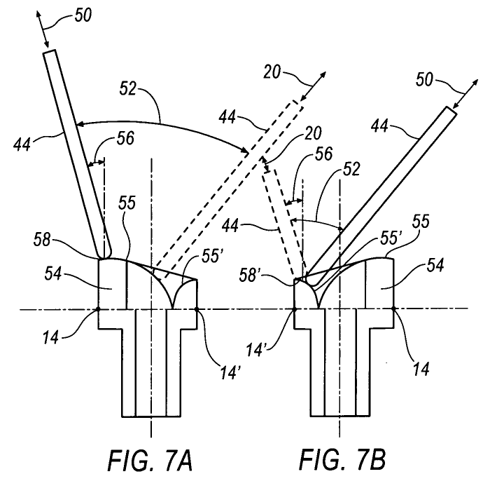

FIGS. 7A and 7B illustrate a cam and follower system according to another embodiment of the present invention. In the illustrated embodiment, the cam and follower system includes a cam body 54 that is substantially similar to cam body 40 described above with at least one exception, namely, cam body 54 includes a smaller cam surface 55, 55′ similar to cam surface 32, 32′ shown in FIG. 4. As with the cam and follower system described above with respect to FIGS. 6A and 6B, the position of follower 44 is movable relative to cam body 54 along an arc 52, albeit with a more limited range of motion due to the smaller cam surface 55, 55′.

Referring still to FIGS. 7A and 7B, a slew angle 56 may be introduced into the position of follower 44 relative to cam body 54 without significantly effecting operation of the cam and follower system. More particularly, slew angle 56 is the angle of follower 44 with respect to cam body 54 that is created by movement of follower 44 about a point 58, 58′ on cam surface 55, 55′, rather than by movement of follower 44 about central surface point 14, 14′. When follower 44 is moved about a point other than central surface point 14, 14′, the stroke length of reciprocating output 50 may change. However, the end of follower 44 that contacts surface 55, 55′ may be configured to reduce the impact of slew angle 56 in the system.

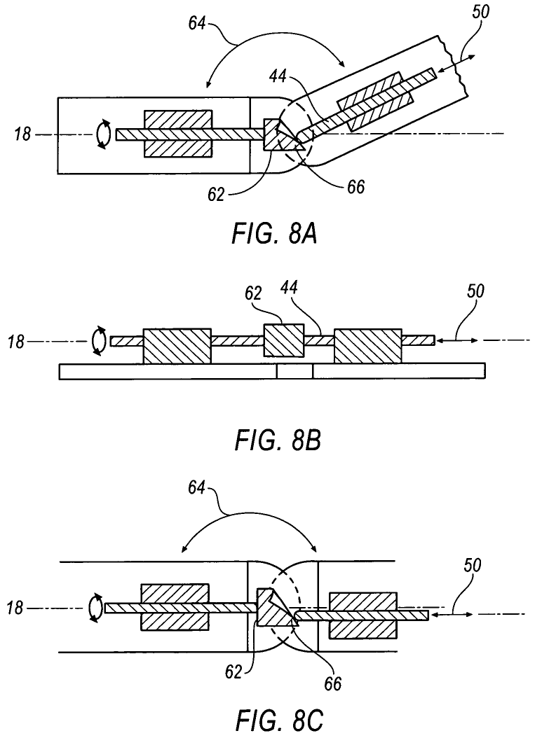

FIG. 8A-8C illustrate a fixture for holding a rotatable cam member 62 and adjustable angle follower 44 according to an embodiment of the present invention. The fixture emphasizes a feature of the present invention in that it allows the output angle 64, 64′ of follower 44 to be changed without significantly changing the stroke length of the reciprocating output 50. Particularly, FIG. 8A is a top view of the cam and follower system, wherein follower 44 is adjusted to an angle 64 relative to the axis of rotation 18 of cam member 62. Angle 64 of follower 44 may be adjusted at a pivot point 66, such as a hinge, which is substantially aligned with the central surface point of the cam surface. When angle 64 is adjusted, the reciprocating output 50 and stroke length remain virtually unchanged. For reference, FIG. 8B illustrates the side view of the fixture described by FIG. 8A.

FIG. 8C illustrates the cam and follower system shown in FIGS. 8A and 8B, wherein the follower 44 is positioned substantially parallel to the axis of rotation 18 and thus, the angle 64′ of follower 44 relative to the axis of rotation 18 of cam member 62 is approximately 180 degrees. While FIGS. 8A-8C generally illustrate the cam and follower configuration shown in FIGS. 7A-7B, the illustrated fixture shown in FIGS. 8A-8C is also applicable to rotatable cam members shown in FIGS. 1-6B.

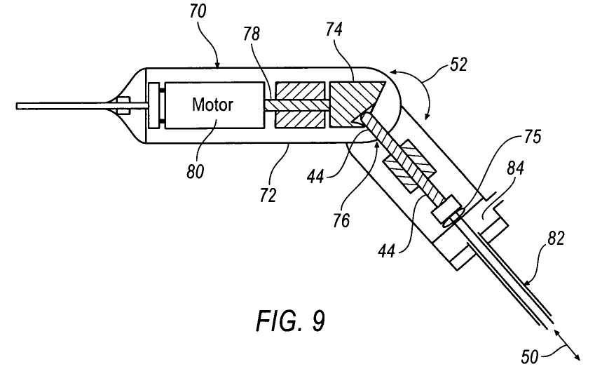

FIG. 9 illustrates the cam and follower system according to an embodiment of the present invention integrated into an adjustable surgical device 70. Surgical device 70 includes a handpiece 72 that provides the necessary support for a cam body 74, a follower 44, a biasing mechanism 75, a hinge 76, which permits the follower angle 52 to be adjusted, and a rotational output 78 coupled to an electric motor 80. The rotational output 78 rotatingly drives cam body 74, which in turn drives follower 44 as described above. Follower 44 is attached to a cutting member 82 that includes an outer cannula and an inner cutting cannula. Follower 44 provides the reciprocating motion to move the inner cutting cannula within the outer cannula. An aspiration line 84 may be connected to a vacuum source for extracting fluids or tissue from a patient. In addition, aspiration line 84 may be used to introduce fluids for anesthesia or irrigation of the surgical site. The cam and follower system allows a surgeon to adjust the angle 52 of the cutting member 82 relative to the remaining of handpiece 72, which improves the ergonomics and control of handpiece 72 and reduces hand fatigue.

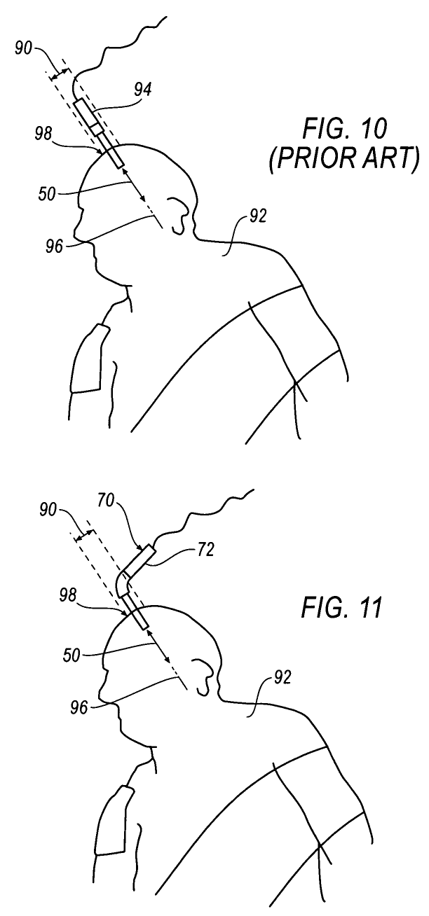

FIG. 10 shows a typical surgical site access region 90 for intracranial surgery on a patient 92. FIG. 10 also illustrates a typical non-adjustable surgical device 94 configured such that the reciprocating output 50 is parallel to the axis of reciprocation 96. The surgical site 98 is substantially blocked by handpiece 94 and hinders the surgeon's view of surgical site 98 or the surgeon's ability to bring other instruments into the region 90.

FIG. 11 illustrates an adjustable surgical device 70 according to an embodiment of the present invention that allows a surgeon to adjust the angle of reciprocating output 50 relative to handpiece 72. In addition to improving the ergonomics of surgical device, the ability to adjust the cutting member 82 relative motor housing portion of the handpiece 72 permits less obscured access into region 90.

The adjustable angle cam and follower system of the present invention, when integrated into a surgical handpiece, improves ergonomics for the surgeon by providing adjustment of the handpiece in relation to the axis of surgical entry. The surgeon is then able to preferentially adjust the angle of the reciprocating output for more comfortable holding of the handpiece. Further, the stability of the handpiece is improved by allowing the surgeon to hold the handpiece body at a preferred angle relative to the axis of surgical entry and improve access into the surgical region during a surgical procedure.

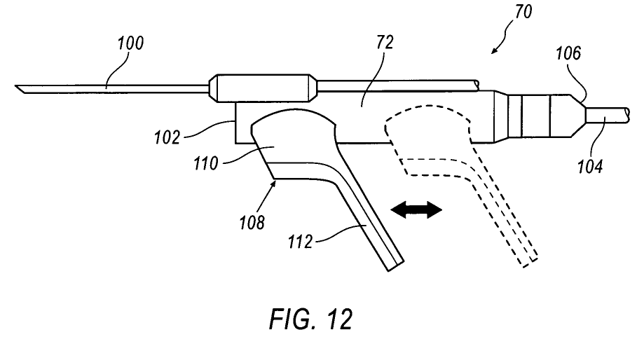

Now referring to FIG. 12, the exemplary surgical device 70 is illustrated. As discussed above, surgical device 70 includes the handpiece 72 and a surgical tool portion 100 that extends from a distal end 102 of handpiece 72. Tool portion 100 may be configured for, but is not limited to, cutting, excising, ablating and irrigating. In the illustrated surgical device 70, a cable 104 extends from a proximal end 106 of handpiece 72 to provide, for example, power, rotational input and/or an aspiration line. FIG. 12 also illustrates an embodiment of an ergonomic attachment 108 particularly, but not necessarily, suited for use with handpiece 72.

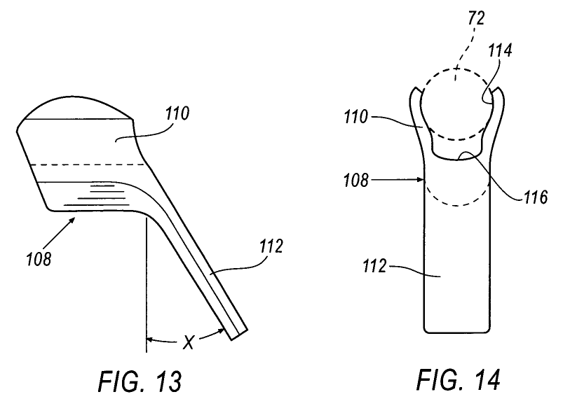

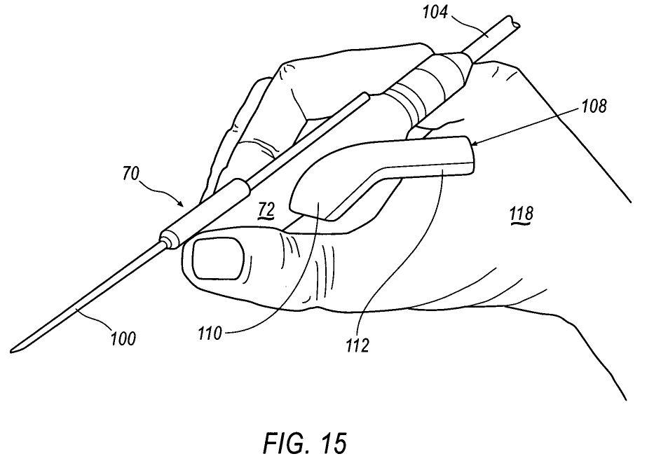

In the illustrated embodiment shown in FIGS. 12-14, ergonomic attachment 108 includes a connecting portion 110 and an extension portion 112. In a particular configuration, connecting portion 110 includes a generally U-shaped groove 114 within which the cylindrical handpiece 72 is received (see, e.g., FIG. 14). The inner surface of receiving groove 114 is generally contoured to closely match the outer surface of handpiece 72. As shown in FIG. 14, ergonomic attachment 108 may include a channel 116 below groove 114 sized to allow passage of an aspiration line, electrical cable and the like.

Ergonomic attachment 108 may be made of a rigid, yet slightly resilient material, such as plastic, which allows connecting portion 110 to be “snapped” onto handpiece 72. The resiliency of the material and/or the contour of the inner surface of groove 114 can be tailored to allow adjustable movement of ergonomic attachment 108 along the axis of handpiece 72 with minimal effort. Optionally, the interface between receiving groove 114 and handpiece 72 may be configured to allow predefined locational placement of ergonomic attachment 108 on handpiece 72. Either or both of connecting portion 110 and handpiece 72 may include, for example, grooves, ridges, bumps, dimples and other suitable features that provide a detent or other feature that creates one or more predefined locational positions. However, other approaches for removably and/or adjustably securing ergonomic attachment 108 to handpiece 72, such as hook-and-loop style fasteners, for example, are also within the scope of present invention.

Ergonomic attachment 108 may be readily removed from surgical device 70 and replaced with a different ergonomic attachment to accommodate a different hand size or preference. Depending on the manner in which connecting portion 110 is affixed to handpiece 72, ergonomic attachment 108 may be removed by sliding connecting portion 110 off of an end of handpiece 72. Alternatively, ergonomic attachment 108 may be removed by applying a pivoting force down and away from handpiece 72, thus providing a “snap off” detachment. In another embodiment, ergonomic attachment 108 may be integrally formed with handpiece 72 and or non-removably attached thereto.

As noted above, ergonomic attachment 108 may be attached by forcing handpiece 72 into groove 114, thus providing a “snap on” attachment. Alternately, ergonomic attachment 108 may be attached to handpiece 72 by sliding connecting portion 110 axially onto handpiece 72.

Extension portion 112 extends from connecting portion 110 at a predetermined angle, creating a shelf-like support that rests on top of a surgeon's hand (see, e.g., FIG. 15). The angle (X) by which extension portion 112 extends from connecting portion 110 can vary depending on the shape and size of the surgeon's hand, and is generally, but not necessarily, in the range of about 0° to 45°.

As shown in FIG. 15, a surgeon may grip handpiece 72 or any other comfortable surface on surgical device 70, including connecting portion 110 of ergonomic attachment 108. Extension portion 112 may be positioned at any point along the length of handpiece 72 from distal end 102 to proximal end 106. Intermediate positions are achieved by sliding connecting portion 110 along handpiece 72. Allowing movement of ergonomic attachment 108 along the length of handpiece 72 enables the surgeon to determine the most comfortable position for ergonomic attachment 108 during the surgical procedure. Ergonomic attachment 108 can be readily moved at any time during a surgical procedure to provide additional comfort or, if desired, removed entirely.

During a surgical procedure, extension portion 112 rests on the surgeon's hand 118 and helps stabilize surgical device 70. By spreading the weight of surgical device 70 over a portion of the surgeon's hand 118, ergonomic attachment 108 removes some of the weight of the surgical device from the surgeon's fingers, which allows the surgeon use of his fingers to maneuver the surgical device with more precision and control. Ergonomic attachment 108 also reduces hand fatigue associated with the surgeon's use of his fingers to support the weight of surgical device 70 and control its movement.

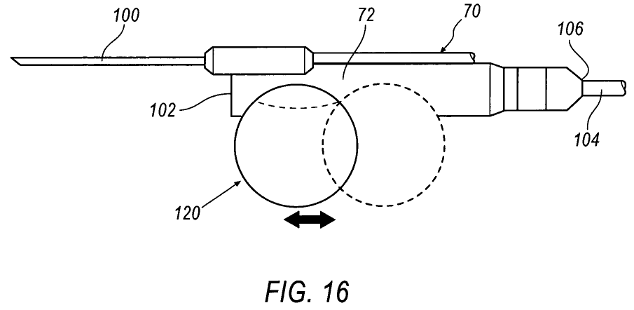

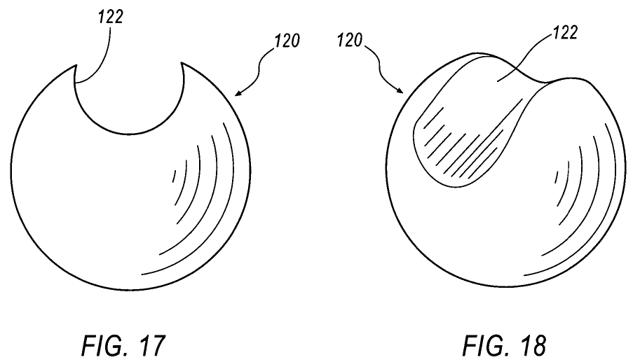

Referring to FIGS. 16-19, another embodiment of the present invention is shown. In this embodiment, an ergonomic attachment 120 is provided that is removably attached to surgical device 70. Ergonomic attachment 120 is generally spherical in shape and includes a groove 122 disposed therein that receives handpiece 72. The inner surface of receiving groove 122 is generally contoured to closely match the contour of the outer surface of handpiece 72.

Ergonomic attachment 120 may be made of a rigid, yet slightly resilient material, such as plastic, which allows it to be “snapped” onto handpiece 72. The resiliency of the material and/or the contour of the inner surface of groove 122 can be tailored to allow adjustable movement of ergonomic attachment 120 along the axis of handpiece 72 with minimal effort. Optionally, the interface between receiving groove 122 and handpiece 72 may be configured to allow predefined locational placement of ergonomic attachment 120 on handpiece 72. Either or both of connecting portion 110 and handpiece 72 may include, for example, grooves, ridges, bumps, dimples and other suitable features that provide a detent or other feature that creates one or more predefined locational positions. However, other approaches for removably securing ergonomic attachment 120 to handpiece 72, such as hook-and-loop style fasteners, for example, are also within the scope of present invention.

Ergonomic attachment 120 may be readily removed from surgical device 70 and replaced with a different ergonomic attachment to accommodate a different hand size or preference. Depending on the manner in which ergonomic attachment 120 is affixed to handpiece 72, ergonomic attachment 120 may be removed by sliding off of an end of handpiece 72 or, alternatively, by applying a pivoting force down and away from handpiece 72, thus providing a “snap off” detachment. In still another embodiment, ergonomic attachment 120 may be integrally formed with handpiece 72 and or non-removably attached thereto.

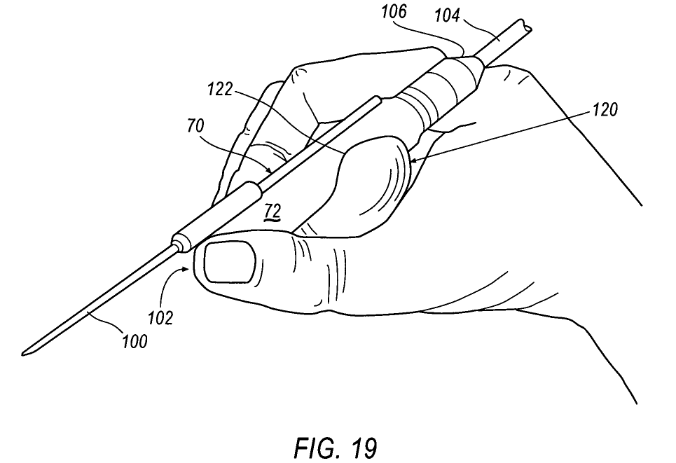

As shown in FIG. 19, a surgeon may grip handpiece 72 or any other comfortable surface on surgical device 70, including ergonomic attachment 120. Ergonomic attachment 120 may be positioned at any point along the length of handpiece 72 from distal end 102 to proximal end 106. Intermediate positions are accomplished by sliding ergonomic attachment 120 along handpiece 72. Allowing movement of ergonomic attachment 120 along the length of handpiece 72 enables the surgeon to decide the most comfortable position for ergonomic attachment 120 during the surgical procedure. Ergonomic attachment 120 can be readily moved at any time during a surgical procedure to provide additional comfort or, if desired, removed entirely. Ergonomic attachment 120 allows the surgeon to support surgical device 70 with all of the fingers on a given hand, removing some or all of the weight of surgical device 70 from his index finger and thumb. This reduction in weight allows the surgeon use of his thumb and/or fingers to maneuver the surgical device with more precision and control.

The present invention has been particularly shown and described with reference to the foregoing embodiments, which are merely illustrative of the best modes for carrying out the invention. It should be understood by those skilled in the art that various alternatives to the embodiments of the invention described herein may be employed in practicing the invention without departing from the spirit and scope of the invention as defined in the following claims. It is intended that the following claims define the scope of the invention and that the apparatus within the scope of these claims and their equivalents be covered thereby. This description of the invention should be understood to include all novel and non-obvious combinations of elements described herein, and claims may be presented in this or a later application to any novel and non-obvious combination of these elements. Moreover, the foregoing embodiments are illustrative, and no single feature or element is essential to all possible combinations that may be claimed in this or a later application.

1. An adjustable cam and follower system for a surgical device comprising:
a tri-dimensional rotatable cam member having a rotational input and a cam surface, the contour of which is defined by at least one radius of varying length; and
a follower selectively moveable linearly, the follower configurable to ride substantially upon the cam surface as the cam rotates.

2. A system according to claim 1, including a hub adapted to connect the cam surface to the rotational input.

3. A system according to claim 1, wherein the cam surface has upper and lower transition surfaces.

4. A system according to claim 3, wherein said upper and lower transition surfaces taper according to said contour of the cam surface.

5. A system according to claim 1, wherein the cam surface does not extend below a predetermined plane.

6. A system according to claim 1, wherein the cam surface does not extend below a predetermined plane and does not extend radially outwardly from a predetermined axis of rotation a distance greater than a predetermined radius.

7. A system according to claim 1, further comprising a handpiece having a proximal and distal end, said tri-dimensional rotatable cam member and said selectively moveable follower being contained within said handpiece.

8. A system according to claim 7 further comprising:
an ergonomic member attachable to said handpiece by sliding a connecting portion of said ergonomic member onto said handpiece.

9. A system according to claim 7 further comprising an ergonomic member integrally formed with said handpiece.

10. A system according to claim 7 further comprising:
an ergonomic member attachable to said handpiece; and
a connecting interface defined by the ergonomic member and said handpiece, the connecting interface being configured to removably or adjustably secure the ergonomic member to the handpiece.

11. A system according to claim 10, wherein said ergonomic member has an extension portion that extends from a connecting portion of the ergonomic member at a predetermined angle.

12. A system according to claim 11, wherein said predetermined angle between said extension portion and said connecting portion is in the range of 0° to 45°.

13. A system according to claim 10, wherein said ergonomic member is generally spherical in shape.

14. A system according to claim 10, wherein the ergonomic member is formed of a plastic material.

15. A fixed displacement adjustable cam and follower system for a surgical device comprising:
a tri-dimensional rotatable cam member having a rotational input and a cam surface defined by at least one radius of varying length;
a selectively moveable follower having a linear output, the follower configured to ride substantially upon the cam surface;
wherein the cam is configured to provide substantially uniform follower displacement for a given movement of the position of the follower relative to the cam member;
a handpiece providing support for said cam member and said follower, wherein said cam member and said follower are contained within said handpiece; and
an ergonomic member having a connecting portion attachable to said handpiece.

16. An adjustable surgical device comprising:
a handpiece having a proximal and distal end;
a hinge positioned between the proximal end and the distal end of the handpiece and configured to allow a first portion of the handpiece to be moved relative to a second portion of the handpiece;
a tri-dimensional rotatable cam member having a rotational input disposed within the proximal end of the handpiece and including a cam surface defined by at least one radius of varying length;
a follower disposed substantially within the distal end of the handpiece and including a proximal end riding upon the cam surface to provide a reciprocating output; and
wherein the cam surface is configured for providing substantially fixed follower displacement as the first handpiece portion is moved relative to the second handpiece portion.

17. A device according to claim 17 further comprising:
an ergonomic member attachable to said handpiece;
a connecting interface defined by the ergonomic member and the surgical device, the connecting interface configured to removably or adjustably secure the ergonomic member to the surgical device.
